(19) United States
(12) Patent Application Publication
(10) Pub. No.: US 2016/0287125 A1
(43) Pub. Date: Oct. 6, 2016

(54) PHYSIOLOGICAL AND PSYCHOLOGICAL CONDITION SENSING HEADSET

(71) Applicant: HEADSENSE MEDICAL LTD., Netanya (IL)

(72) Inventors: Guy WEINBERG, Closter, NJ (US); Surik PAPYAN, Haifa (IL)

(21) Appl. No.: 15/037,448

(22) PCT Filed: Nov. 19, 2014
(86) PCT No.: PCT/IL2014/051003

§ 371 Date: May 18, 2016

Related U.S. Application Data
(60) Provisional application No. 61/911,014, filed on Dec. 3, 2013.

(51) Int. Cl. A61B 5/0478; A61B 5/0205; A61B 5/00; A61B 5/04

(52) U.S. Cl. CPC A61B 5/0478; A61B 5/04017; A61B 5/02055; A61B 5/742; A61B 5/002; A61B 5/4872; A61B 5/6803; A61B 2562/0204; A61B 5/0816

(57) ABSTRACT

A user physiological condition sensing apparatus constituted of: a thermal sensor, the thermal sensor arranged to be positioned on the neck of the user and sense the skin temperature of the user; a core temperature functionality in communication with the thermal sensor, the core temperature functionality arranged to determine the core temperature of the user responsive to the sensed skin temperature; and an output module in communication with the core temperature functionality, the output module arranged to output the determined user core temperature, wherein the core temperature determination is further responsive to the weight and height of the user.

### TECHNICAL FIELD

The present invention relates generally to the field of physiological and psychological condition sensing.

### BACKGROUND

Exercising is a common activity performed worldwide. Unfortunately, when exercising a user is not always provided with useful exercise information therefore the workout is less than optimal. Additionally, while exercising, users generally become bored as they have nothing to do with their minds. Thus, there is a long felt need for a system able to provide useful exercise information and activity for the mind.

### SUMMARY

Accordingly, it is a principal object to overcome at least some of the disadvantages of prior art. This is accomplished in certain embodiments by providing a user physiological condition sensing apparatus comprising: an acoustic sensor, the acoustic sensor arranged to be positioned so as to sense sound waves emitting from a carotid artery of the user and sound waves emitting from the trachea of the user; a heart rate functionality in communication with the acoustic sensor and the output module, the heart rate functionality arranged to determine the heart rate of the user responsive to the sensed carotid artery emitted sound waves; a respiratory rate functionality in communication with the acoustic sensor and the output module, the respiratory rate functionality arranged to determine the respiratory rate of the user responsive to the sensed trachea emitted sound waves; and an output module in communication with the heart rate functionality and the respiratory rate functionality, the output module arranged to output the determined heart rate and respiratory rate.

In one embodiment, the apparatus further comprises: a thermal sensor, the thermal sensor arranged to be positioned on the neck of the user and sense the skin temperature of the user; and a core temperature functionality in communication with the thermal sensor, the core temperature functionality arranged to determine the core temperature of the user responsive to the sensed skin temperature, the output module further arranged to output the determined user core temperature, wherein the core temperature determination is further responsive to the weight and height of the user.

Independently, embodiments provide for a user psychological condition sensing apparatus, the apparatus comprising: a plurality of electroencephalography (EEG) electrodes arranged to be positioned on the head of the user and arranged to receive EEG signals; an output module; a user input device; a psychological application functionality in communication with the output module and the user input device, the psychological application functionality arranged to provide to the output module an interactive psychological application arranged to be output to the user, the psychological application functionality arranged to receive input from the user regarding the interactive psychological application via the user input device; and a psychological condition functionality in communication with the EEG electrodes and the psychological application functionality, the psychological condition functionality arranged to determine a psychological condition of the user responsive to the received EEG signals and the received user input.

In one embodiment, the apparatus further comprises: a concentration functionality in communication with the EEG electrodes, the concentration functionality arranged to compare the intensity of EEG signal received by the EEG electrodes over a predetermined time period with a predetermined value; and a concentration control module, the concentration control module arranged to output a concentration control signal exhibiting a first state when the received EEG signals are greater than the predetermined value and output a concentration control signal exhibiting a second state when the received EEG signals are not greater than the predetermined value, the second state different than the first state.

Independently, embodiment provide for a method of sensing a user physiological condition, the method comprising: sensing sound waves emitting from a carotid artery of the user and sound waves emitting from the trachea of the user;

determining the heart rate of the user responsive to the sensed carotid artery emitted sound waves; determining the respiratory rate of the user responsive to the sensed trachea emitted sound waves; and outputting the determined heart rate and respiratory rate.

In one embodiment the method further comprises: determining the rate of calorie burning by the user responsive to the determined heart rate; and outputting the determined calorie burning rate. In another embodiment the method further comprises determining a plurality of exercise zones responsive to the determined user heart rate. In yet another embodiment the method further comprises: providing a rest heart rate database; storing on the provided rest heart rate database the determined user heart rate when the user is resting; and outputting a history of user heart rate when at rest.

In one embodiment the method further comprises: sensing the skin temperature of the user; determining the core temperature of the user responsive to the sensed skin temperature and further responsive to the weight and height of the user; and outputting the determined user core temperature. In another embodiment the method further comprises sensing the electrical resistance of a skin of the user; determining the humidity within the skin of the user; and outputting the determined humidity.

Independently, embodiments provide for a method of sensing a user psychological condition, the method comprising: receiving electroencephalography (EEG) signals of the user; receiving input from the user regarding an interactive psychological application; and determining a psychological condition of the user responsive to the received EEG signals and the received user input.

In one embodiment the method further comprises: comparing the intensity of the received EEG signals over a predetermined time period with a predetermined value; and outputting a concentration control signal exhibiting a first state when the received EEG signals are greater than the predetermined value and output a concentration control signal exhibiting a second state when the received EEG signals are not greater than the predetermined value, the second state different than the first state.

Additional features and advantages will become apparent from the following drawings and description.

### BRIEF DESCRIPTION OF THE DRAWINGS

For a better understanding of the invention and to show how the same may be carried into effect, reference will now be made, purely by way of example, to the accompanying drawings in which like numerals designate corresponding elements or sections throughout.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the accompanying drawings:

FIG. 1 illustrates a high level schematic diagram of a user physiological condition sensing apparatus comprising an acoustic sensor, according to certain embodiments;

FIG. 2 illustrates a high level schematic diagram of the user physiological condition sensing apparatus of FIG. 1, further comprising a thermal sensor, according to certain embodiments;

FIG. 3 illustrates a high level schematic diagram of the user physiological condition sensing apparatus of FIG. 1, further comprising an electrical resistance sensor;

FIG. 4 illustrates a high level schematic diagram of a user psychological condition sensing apparatus, according to certain embodiments;

FIG. 5 illustrates a high level schematic diagram of a combined user physiological and psychological condition sensing apparatus, according to certain embodiments;

FIG. 6 illustrates a high level flow chart of a method of sensing physiological conditions of a user, according to certain embodiments; and

FIG. 7 illustrates a high level flow chart of a method of sensing psychological conditions of a user, according to certain embodiments.

### DETAILED DESCRIPTION OF THE PREFERRED EMBODIMENTS

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

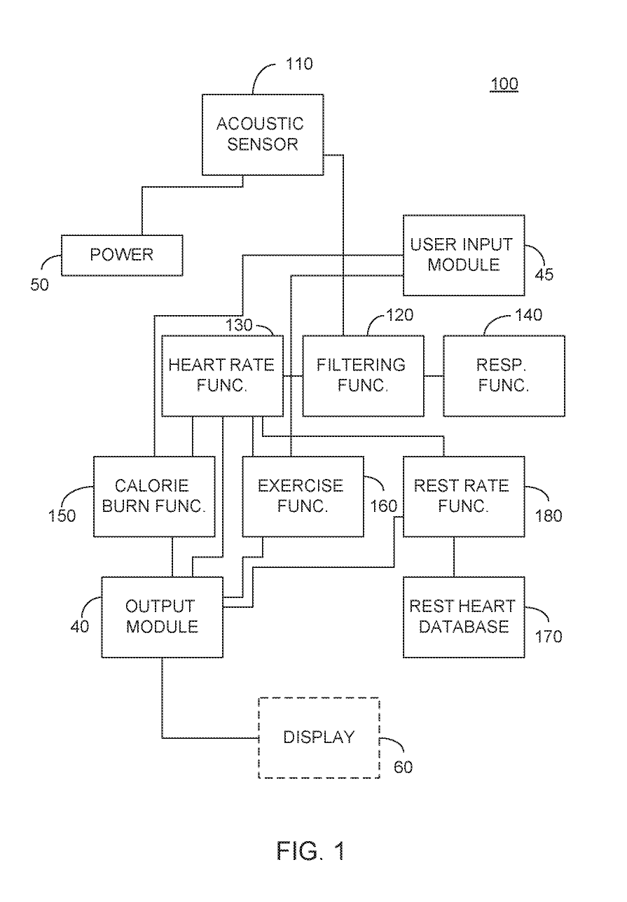

FIG. 1 illustrates a high level schematic diagram of a user physiological condition sensing apparatus 100. User physiological condition sensing apparatus 100 comprises: an output module 40; a user input module 45; a power source 50; an optional display 60; an acoustic sensor 110; a filtering functionality 120; a heart rate functionality 130; a respiratory rate functionality 140; a calorie burning rate functionality 150; an exercise functionality 160; a rest heart rate database 170; and a rest rate functionality 180. Filtering functionality 120 is in communication with acoustic sensor 110, heart rate functionality 130 and respiratory rate functionality 140. Heart rate functionality 130 and respiratory rate 140 are each further in communication with output module 40, calorie burning rate functionality 150, exercise functionality 160 and rest rate functionality 180. Filtering functionality 120, heart rate functionality 130, respiratory functionality 140, calorie burning rate functionality 150, exercise functionality 160 and rest rate functionality 180 can each be implemented by any of: a dedicated functionality; computer readable instructions for a general purpose computing device or processor, the readable instructions stored on a memory; dedicated hardware; and a dedicated control circuitry, without limitation.

In one embodiment, power source 50 comprises a battery. In another embodiment, power source 50 comprises a rechargeable power source. Power source 50 provides power to the components of user physiological condition sensing apparatus 10. Output module 40 is in communication with optional display 60. In another embodiment, output module 40 is in wireless communication with a mobile device. In one further embodiment, output module 40 is in Bluetooth communication with a smartphone.

Acoustic sensor 110 is positioned on the neck of the user such that sound waves emitted from a carotid artery of the user and sound waves emitted from the trachea of the user are sensed by acoustic sensor 110. In particular, acoustic sensor 110 is arranged to sense the sound waves emitted by the systole and diastole of the heart and the sound waves emitted during inhalation, expiration, wheezing and snoring.

In operation, filtering functionality 120 is arranged to validate that the received acoustic signal is strong enough for further analysis. In particular, the body generates physiological acoustic signals, such as heart beats, breathing sounds, muscle movements, stomach movements, blood flow, etc. The external environment generates noise such as talking, music, walking steps, etc. Filtering functionality 120 is arranged to apply a signal saturation filter on the received acoustic signal of acoustic sensor 110 and apply a signal to noise ration test on the filtered signal to determine if the intensity of the heart and respiratory acoustic signals are at least twice as strong as the background noise. The signal saturation filter is arranged to filter out saturations of acoustic sensor 110 which may be caused by acoustic sensor 110 not being firmly attached to the neck or by a loud external noise.

If the signal passes the quality test, filtering functionality 120 is arranged to extract the frequencies associated with the heart and the frequencies associated with the respiratory system. In one embodiment, a Fourier transform is performed on the signal. Optionally, a low pass filter is applied to the signal to filter out all frequencies above 4 Hz. A first band pass filter (BPF) is arranged to extract the frequencies associated with cardiac systole and diastole and a second BPF is arranged to extract the frequencies associated with the respiratory inhalation and exhalation. The frequency pass range of the first BPF is 0.833-3 Hz, since the heart rate varies between 50-180 beats per minute. The frequency pass range of the second BPF is 0.15-0.4 Hz, since the respiratory cycle varies between 8-20 cycles per minute. An inverse Fourier transform is performed on the extracted frequencies and a peak identification module is applied on the signals so as to determine the heart rate and respiratory rate in beats per minutes and breaths per minute, respectively. In particular, the heart rate is determined by detecting the distance between peaks of adjacent diastole segments and the respiratory rate is determined by detecting the distance between peaks of adjacent exhalation segments.

Calorie burning rate functionality 150 is arranged to determine the rate of calorie burning by the user. In particular, calorie burning rate functionality is arranged to determine the calorie burning rate responsive to the determined heart rate of the user and the weight and age of the user entered on user input module 45. The calculations for determining the calorie burning rate are described in an article titled “Prediction of energy expenditure from heart rate monitoring during submaximal exercise” by Keytel L. R. et al., published in the Journal of Sports Sciences, 2005, the entire contents of which are incorporated herein by reference. In particular, the calorie burning rate, per minute, for men is given as:

C/min=(−55.09+0.63*HR+0.198*weight+0.2*age)/4.184 EQ. 1

where HR is the user heart rate, weight is the user's weight and age is the user's age.

The calorie burning rate, per minute, for women is given as:

C/min=(−20.4+0.44*HR+0.126*weight+0.07*age)/4.184 EQ. 2

The determined calorie burning rate is output by output module 40, optionally to optional display 60.

Exercise functionality 160 is arranged to determine a plurality of exercise zones responsive to the determined heart rate of the user, and the entered age and gender of the user. In particular, each exercise zone is associated with a different type of exercise. In one embodiment, the plurality of exercise zones comprises: a moderate activity zone, associated with warm ups or simple work outs; a weight control zone, associated with fat burning and fitness; an aerobic zone, associated with cardiac work out; an anaerobic zone, i.e. exercise intense enough to trigger lactic acid formation; and a maximum work out zone. Each zone is determined as a percentage of the maximum heart rate of the user. For example, the user is exercising in: the moderate activity zone when his heart rate is 50%-60% of his maximum heart rate; the weight control zone when his heart rate is 60%-70% of his maximum heart rate the aerobic zone when his heart rate is 70%-80% of his maximum heart rate; the anaerobic activity zone when his heart rate is 80%-90% of his maximum heart rate; and the maximum workout zone when his heart rate is 90%-100% of his maximum heart rate. In one embodiment, the different zones are displayed to the user, along with his determined heart rate, so the user can see the particular exercise zone he is working out in. If the user wishes to improve his abilities in a certain area, he should repeatedly work out in that particular zone. In one embodiment, the maximum heart rate for men, in beats per minute, is given as:

maxHR=220−age EQ. 3

The maximum heart rate for women, in beats per minute, is given as:

maxHR=226−age EQ. 4

Rest rate functionality 180 is arranged to store on rest heart rate database 170 the user heart rate determined by heart rate functionality 130 when the user is resting, such that a history of user heart rates when at rest is stored on heart rate database 170. Output module 40 is arranged to output the stored user heart rate history, optionally to optional display 60. In one embodiment, rest rate functionality 180 is further arranged to compare the current determined heart rate with heart rates stored on heart rate database 170 and output module 40 is arranged to output the comparison.

In one embodiment, output module 40 is further arranged to output to optional display 60 exercise tips from a database of exercise tips and explanations associated with different types of exercises and work out goals.

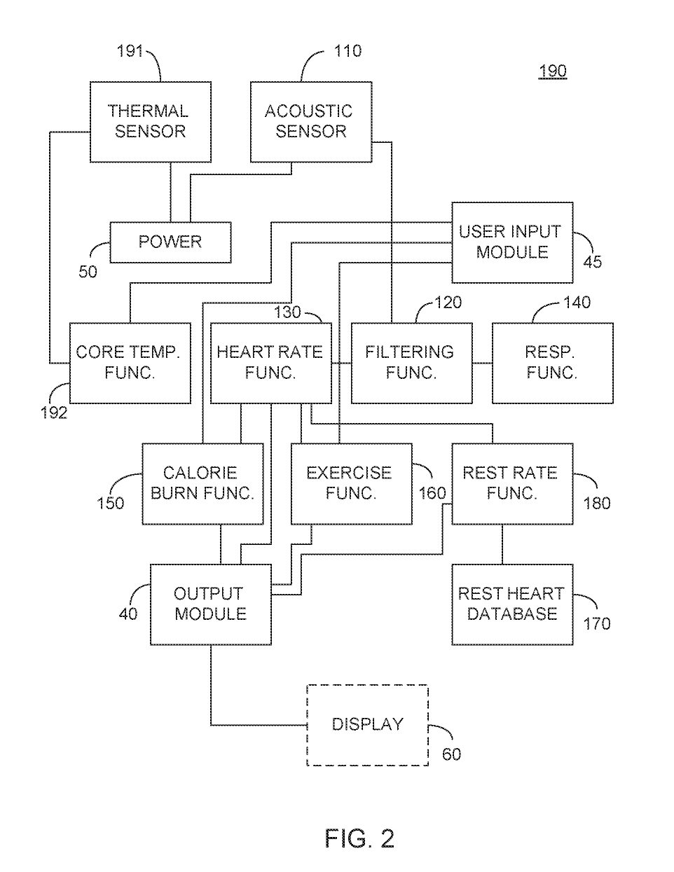

FIG. 2 illustrates a high level schematic diagram of a user physiological condition sensing apparatus 190. User physiological condition sensing apparatus 190 is in all respects similar to user physiological condition sensing apparatus 100 of FIG. 1, with the addition of: a thermal sensor 191; and a core temperature functionality 192. Thermal sensor 191 is positioned on the neck of a user, in one embodiment below the ear. In one particular embodiment, thermal sensor 191 is positioned to juxtapose the carotid artery of the user. In one embodiment, two thermal sensors 191 are provided and positioned on opposing sides of the user's neck. Core temperature functionality 192 can be implemented by any of: a dedicated functionality; computer readable instructions for a general purpose computing device or processor, the readable instructions stored on a memory; dedicated hardware; and a dedicated control circuitry, without limitation. In another embodiment, core temperature functionality 192 and output module 40 can be provided as a single functionality, without exceeding the scope. Thermal sensor 191 is in communication with core temperature functionality 192. Core temperature functionality 192 is further in communication with output module 40 and user input module 45.

In operation, the user is prompted to enter at user input module 45 his age, weight and height. Thermal sensor 191 is arranged to sense the skin temperature at the neck of the user. Core temperature functionality 192 is arranged to determine the core temperature of the user responsive to the sensed skin temperature and the input user information. In particular, the core temperature is the temperature of the blood within the carotid artery. The difference between the core temperature and the skin temperature is caused by muscle, fat and skin tissue. These play a main role in isolating the core temperature from the outside environment and thus also have an isolating effect between the core temperature and the skin temperature. In particular, a user with a higher fat percentage will exhibit a greater difference between core temperature and skin temperature. The input user information is utilized to determine a function of the user fat percentage. In one embodiment, core temperature functionality 192 is arranged to determine the body mass index (BMI) of the user. Responsive to the determined user BMI, core temperature functionality 192 is further arranged to determine a heat transfer coefficient of the user's neck. Core temperature functionality 192 is then arranged to determine the user core temperature responsive to the sensed skin temperature and the determined heat transfer coefficient. Output module 40 is arranged to output the determined core temperature, optionally to optional display 60.

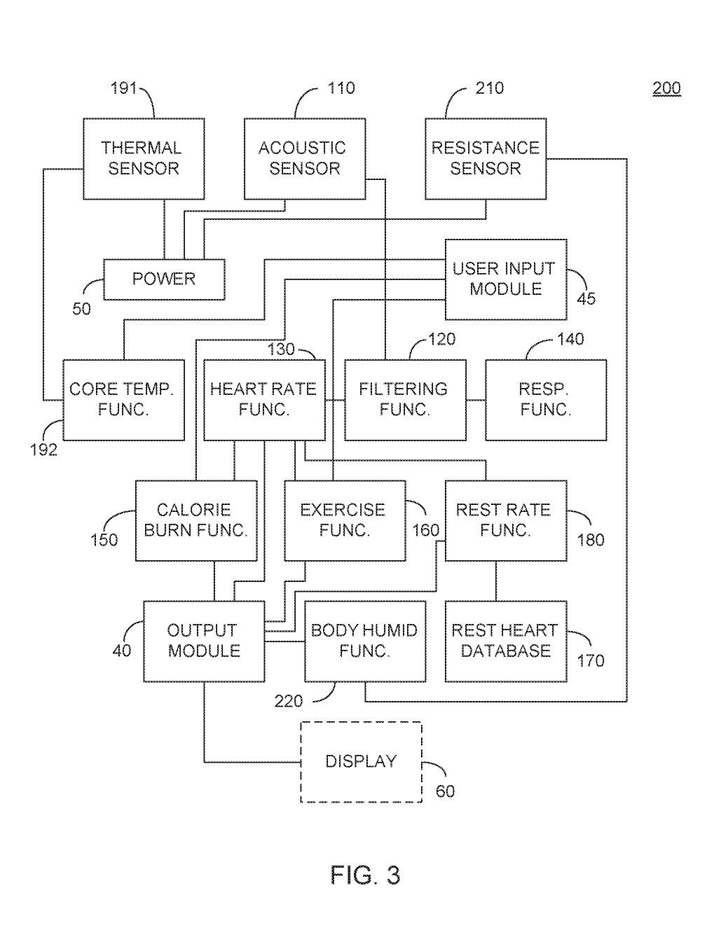

FIG. 3 illustrates a high level schematic diagram of a user physiological condition sensing apparatus 200. User physiological condition sensing apparatus 200 is in all respects similar to user physiological condition sensing apparatus 100 of FIG. 2, further comprising: an electrical resistance sensor 210; and a body humidity functionality 220. Body humidity functionality 220 can be implemented by any of: a dedicated functionality; computer readable instructions for a general purpose computing device or processor, the readable instructions stored on a memory; dedicated hardware; and a dedicated control circuitry, without limitation. Electrical resistance sensor 210 is positioned on the neck of the user and is in communication with body humidity functionality 220. Body humidity functionality 220 is further in communication with output module 40.

In operation, electrical resistance sensor 210 is arranged to provide a current within the skin of the user and measure the resistance of the skin. In one embodiment, the provided current is generated by an internal power source and in another embodiment the provided current is generated by power source 50. Body humidity functionality is arranged to determine the percentage of humidity within the user skin responsive to the determined electrical resistance of the skin. In particular, the humidity of the skin causes a change in the electrical resistance, as humid skin provides less electrical resistance than dry skin.

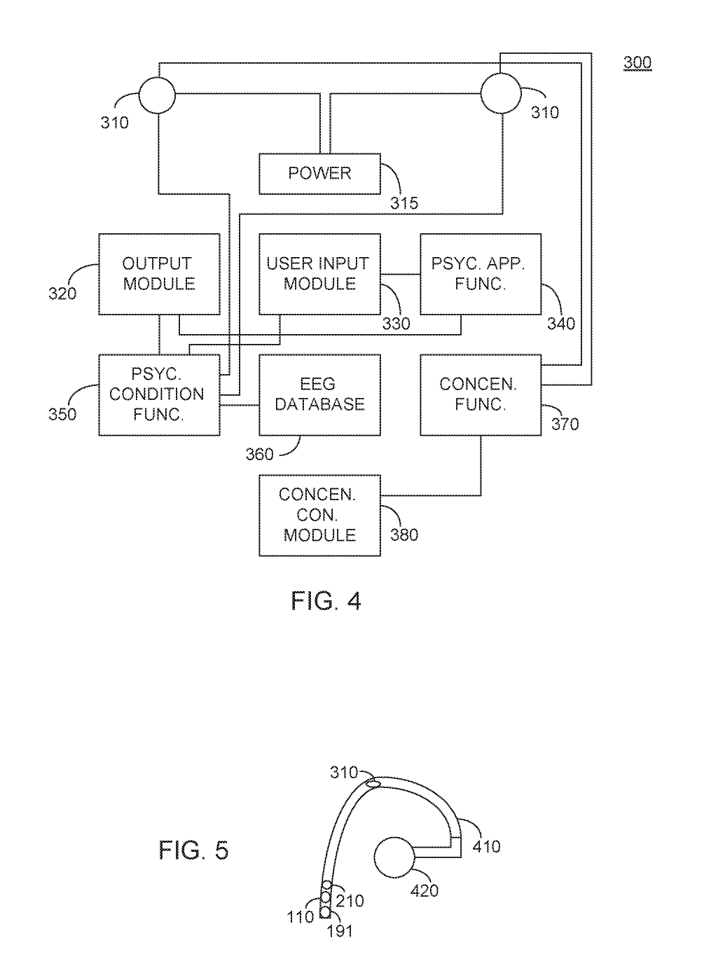

FIG. 4 illustrates a high level schematic diagram of a user psychological condition sensing apparatus 300, comprising: a plurality of electroencephalography (EEG) electrodes 310; a power source 315; an output module 320; a user input module 330; a psychological application functionality 340; a psychological condition functionality 350; an EEG database 360; a concentration functionality 370; and a concentration control module 380. In one embodiment, power source 315 comprises a battery and in another embodiment power source 315 comprises a rechargeable power source. Psychological application functionality 340, psychological condition functionality 350, concentration functionality 370 and concentration control module 380 can each be implemented by any of: a dedicated functionality; computer readable instructions for a general purpose computing device or processor, the readable instructions stored on a memory; dedicated hardware; and a dedicated control circuitry, without limitation. Output module 320 is in communication with a display 360. In one embodiment, display 360 is part of a mobile device. In one further embodiment, output module 320 is in Bluetooth communication with the display 360 of a smartphone. EEG electrodes 310 are in communication with power source 315 and are powered thereby. Psychological application functionality 340 is in communication with output module 320, user input module 330 and psychological condition functionality 350. Psychological condition functionality 350 is further in communication with EEG electrodes 310, user input module 330, output module 320 and EEG database 360. In one embodiment, two EEG electrodes 310 are provided and positioned on opposing sides of the user's head.

EEG database 360 has stored thereon a plurality of EEG signals associated with different psychological states, as will be described below. Concentration functionality 370 is in communication with EEG electrodes 310 and concentration control module 380. Concentration control module 380 is in communication with an external device, such as a smartphone.

In operation, psychological application functionality 340 is arranged to output, via output module 320, a request for the user to select a particular psychological test. The user selects the particular test via user input module 330 and psychological application functionality 340 is arranged to output the test to the user. In one embodiment, the selection and running of the test are accomplished on the user's smartphone. Psychological condition functionality 350 is arranged to analyze the test results and determine a particular psychological state of the user responsive thereto. In particular, in one embodiment several psychological state may be determined, each associated with a particular psychological test.

A first state is a concentration state determining the level of concentration of the user. A test of variables of attention (T.O.V.A.) is selected and performed by the user. The T.O.V.A. measures the attention state of the user, i.e. how well the user can pay attention, by measuring responses to audio or video stimuli. Psychological condition functionality 350 is further arranged to compare EEG signals received by EEG sensors 310 while taking the test with EEG signals stored on EEG database 360 associated with different concentration levels. The comparison with stored EEG signals provides indication of the current concentration level of the user. Psychological condition functionality 350 is further arranged to determine the concentration level of the user responsive to both the T.O.V.A. and the EEG signal comparison.

A second state is a memory state determining the memory quality of the user. A Phonological Awareness Test 2 (PAT2) is selected and performed by the user. The PAT2 assesses the user's awareness of the oral language segments that comprise words, i.e. syllables and phonemes. Psychological condition functionality 350 is further arranged to compare EEG signals received by EEG sensors 310 while taking the test with EEG signals stored on EEG database 360 associated with different memory conditions. The comparison with stored EEG signals provides indication of the current condition of the memory of the user. Psychological condition functionality 350 is further arranged to determine the attention state of the user responsive to both the T.O.V.A. and the EEG signal comparison.

A third state is the awareness state determining how aware the user is of his surroundings.

A fourth state is the mood state determining the current mood of the user. In particular, EEG signals from the limbic system are analyzed to determine the mood of the patient. The limbic system supports a wide variety of functions, including: emotion; behavior; motivation; long term memory; and olfaction. The received EEG signals are compared to EEG signals stored in EEG database 360 associated with different moods, the comparison with the stored EEG signals providing information regarding the current mood of the user.

In all cases, output module 320 is arranged to output the determined psychological condition of the user.

Concentration functionality 370 is arranged to analyze the intensity of the received EEG signals. In the event the intensity of the EEG signals over a predetermined time period are greater than a predetermined value, concentration control module is arranged to output a concentration control signal exhibiting a first state. In the event the intensity of the EEG signals over a predetermined time period are not greater than the predetermined value, concentration control module is arranged to output a concentration control signal exhibiting a second state, opposing the first state. In particular, the first state of the concentration control signal is arranged to perform a first action on the external device and the second state of the concentration control signal is arranged to perform a second action, different than the first action. Thus, the external device is controlled by the level of concentration of the user, because the more the user concentrates the greater the intensity of the EEG signals become.

FIG. 5 illustrates a high level schematic diagram of a combined user physiological and psychological condition sensing apparatus 400. Apparatus 400 combines user physiological condition sensing apparatus 200 of FIG. 3 and user psychological condition sensing apparatus 300 of FIG. 4 and thus in the sake of brevity will not be further described. Advantageously, thermal sensor 191, acoustic sensor 110, electrical resistance sensor 210 and EEG electrodes 310 are disposed on a headset 410 comprising an ear phone 420, which is comfortably worn by the user. Due to the shape of headset 410, thermal sensor 191, acoustic sensor 110 and resistance sensor 210 are positioned near the carotid artery of the user. Additionally, EEG sensor 310 is positioned on the user's head.

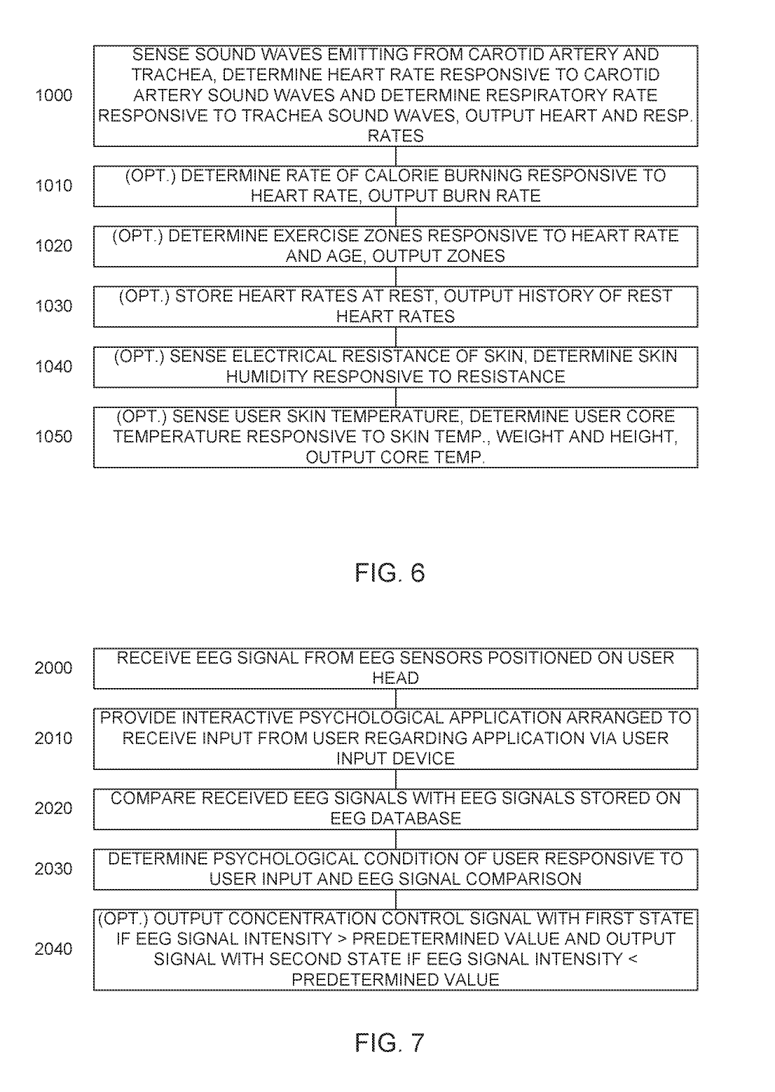

FIG. 6 illustrates a high level flow chart of a method of sensing physiological conditions of a user. In stage 1000, sound waves emitted from the carotid artery and trachea are sensed. The heart rate of the user is determined responsive to the frequencies associated with sound waves of the carotid artery and the respiratory rate of the user is determined responsive to the frequencies associated with sound waves of the trachea. The determined heart rate and respiratory rate is then output by an output module.

In optional stage 1010, the rate of calorie burning of the user responsive to the determined heart rate of stage 1000. The determined user calorie burning is then output by the output module of stage 1000. In optional stage 1020, exercise zones are determined responsive to the determined heart rate of stage 1000 and responsive to the age of the user. The determined exercise zones are output to a display. In optional stage 1030, the determined heart rates of the user of stage 1000, when at rest, are stored on a database. A history of user heart rates when at rest is output by the output module of stage 1000. In optional stage 1040, the electrical resistance of the user's skin is sensed. The humidity of the skin is determined responsive to the sensed resistance of the skin and output by the output module of stage 1000.

In optional stage 1050, the skin temperature of the user is sensed. The core temperature of the user is determined responsive to the sensed skin temperature and further responsive to the weight and height of the user. The determined core temperature is output by the output module of stage 1000.

FIG. 7 illustrates a high level flow chart of a method of sensing psychological conditions of a user. In stage 2000, EEG signals are received from EEG sensors placed on the head of the user. In stage 2010, an interactive psychological application is provided to the user and user input regarding the interactive psychological application is received at a user input module. In stage 2020, the received EEG signals of stage 2000 are compared with EEG signals stored on an EEG database, the stored EEG signals each associated with a particular psychological state. In stage 2030, a psychological condition of the user is determined responsive to the received user input of stage 2010 and responsive to the comparison of stage 2020. In optional stage 2040, the intensity of the received EEG signals of stage 2000 over a predetermined time period are compared to a predetermined time period. In the event the EEG signal intensity is greater than a predetermined value, a concentration control signal exhibiting a first state is output. In the event the EEG signal intensity is not greater than a predetermined value, a concentration control signal exhibiting a second state is output, the second state opposing the first state. The output concentration control signal is arranged to control an external device.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. In the claims of this application and in the description of the invention, except where the context requires otherwise due to express language or necessary implication, the word “comprise” or variations such as “comprises” or “comprising” is used in any inclusive sense, i.e. to specify the presence of the stated features but not to preclude the presence or addition of further features in various embodiments of the invention.

Unless otherwise defined, all technical and scientific terms used herein have the same meanings as are commonly understood by one of ordinary skill in the art to which this invention belongs. Although methods similar or equivalent to those described herein can be used in the practice or testing of the present invention, suitable methods are described herein.

All publications, patent applications, patents, and other references mentioned herein are incorporated by reference in their entirety. In case of conflict, the patent specification, including definitions, will prevail. In addition, the materials, methods, and examples are illustrative only and not intended to be limiting. No admission is made that any reference constitutes prior art. The discussion of the reference states what their author's assert, and the applicants reserve the right to challenge the accuracy and pertinency of the cited documents. It will be clearly understood that, although a number of prior art complications are referred to herein, this reference does not constitute an admission that any of these documents forms part of the common general knowledge in the art in any country.

It will be appreciated by persons skilled in the art that the present invention is not limited to what has been particularly shown and described hereinabove. Rather the scope of the present invention is defined by the appended claims and includes both combinations and sub-combinations of the various features described hereinabove as well as variations and modifications thereof, which would occur to persons skilled in the art upon reading the foregoing description.

1-6. (canceled)

7. A user psychological condition sensing apparatus, comprising:
a plurality of electroencephalography (EEG) electrodes arranged to be positioned on the head of the user and arranged to receive EEG signals;
an output module;
a user input device;
a psychological application functionality in communication with said output module and said user input device, said psychological application functionality arranged to provide to said output module an interactive psychological application arranged to be output to the user, said psychological application functionality arranged to receive input from the user regarding the interactive psychological application via said user input device; and
a psychological condition functionality in communication with said EEG electrodes and said psychological application functionality, said psychological condition functionality arranged to determine a psychological condition of the user responsive to said received EEG signals and said received user input.

8. The apparatus of claim 7, further comprising:
a concentration functionality in communication with said EEG electrodes, said concentration functionality arranged to compare the intensity of EEG signal received by said EEG electrodes over a predetermined time period with a predetermined value; and
a concentration control module, said concentration control module arranged to output a concentration control signal exhibiting a first state when said received EEG signals are greater than the predetermined value and output a concentration control signal exhibiting a second state when said received EEG signals are not greater than the predetermined value, said second state different than said first state.

9-14. (canceled)

15. A method of sensing a user psychological condition, the method comprising:
receiving electroencephalography (EEG) signals of the user;
receiving input from the user regarding an interactive psychological application; and
determining a psychological condition of the user responsive to said received EEG signals and said received user input.

16. The method of claim 15, further comprising:
comparing the intensity of the received EEG signals over a predetermined time period with a predetermined value; and
outputting a concentration control signal exhibiting a first state when said received EEG signals are greater than the predetermined value and output a concentration control signal exhibiting a second state when said received EEG signals are not greater than the predetermined value, said second state different than said first state.
